(19) United States
(12) Patent Application Publication
(10) Pub. No.: US 2017/0260568 A1
(43) Pub. Date: Sep. 14, 2017

(54) Method For The Colorimetric Detection Of The Amplification Of A Target Nucleic Acid Sequence

(71) Applicant: Fondazione Istituto Italiano di Tecnologia, Genova (IT)

(72) Inventors: Pier Paolo Pompa, Lecce (IT); Paola Valentini, Genova (IT)

(73) Assignee: Fondazione Istituto Italiano di Tecnologia, Genova (IT)

(21) Appl. No.: 15/529,858

(22) PCT Filed: Dec. 11, 2015
(86) PCT No.: PCT/IB2015/059546

§ 371 Date: May 25, 2017

(30) Foreign Application Priority Data
Dec. 15, 2014 (IT) TO2014A001037

(51) Int. Cl. C12Q 1/68

(52) U.S. Cl. CPC C12Q 1/686; C12Q 1/6834; C12Q 1/6825; C12Q 1/6823

(57) ABSTRACT

The invention relates to methods and kits for the detection of the amplification of a target nucleic acid. The methods and kits of the invention are based on the use of colloidal gold nanoparticles functionalized with a first and a second oligonucleotide probe, whose sequences are designed on a universal TAG sequence that is incorporated in the amplification product.

The present invention relates to a method for the detection of the amplification of a target nucleic acid sequence in a liquid sample in which the presence of an amplification product is determined colorimetrically, by exploiting the known property of colloidal gold nanoparticles (AuNPs) to give rise to a visible colour change when, as a result of their aggregation, the interparticle distance becomes less than the size (diameter) of the particles (an effect due to plasmon coupling).

Nucleic acid amplification reactions, and in particular the PCR, are an extremely powerful tool that has revolutionized the molecular biology field in the last thirty years. These techniques allow for the rapid and sensitive identification of a target nucleic acid of interest, even if present in the sample in very low concentrations or diluted in an excess of interfering genetic material. The PCR, and the other known techniques for nucleic acid amplification allow to detect the presence of infectious agents or gene mutations or rearrangements in samples obtained from patients, or to detect the presence of pathogenic contaminants in food or water samples. They also find wide application in the scientific research field and are fundamental for the screening of populations of laboratory animals in order to identify transgenic subjects.

The standard procedure for the verification of the results of a PCR or other nucleic acid amplification technique comprises performing an agarose gel electrophoresis, staining the gel, and finally reading it with a scanner or reader equipped with an excitation lamp at an appropriate wavelength. The working time required for the entire procedure (which entails the preparation of the gel, the electrophoretic run, the staining and washing, the reading, and the interpretation of the results) is estimated at around at least 2-3 hours. The standard agarose gels allow for the analysis of up to 19 samples at a time, therefore, if there is a greater number of samples, the entire procedure must be performed on multiple gels in parallel, which multiplies costs and equipment, or it must be repeated several times, with a considerable increase in working times. Even if these procedures are extremely standardized, they still require expensive reagents, long processing times, and specific instrumentation. These methodologies can also become difficult to carry out in some high throughput applications, such as for example the identification of transgenic animals, in which a reduction in the time of analysis would have a major impact on cost reduction and mitigation of the suffering inflicted on the animals themselves, as well as in the clinical diagnostics field, in which emergency screenings for pathogens causing epidemics may be required.

Cross-linking experiments for colloidal gold nanoparticles (AuNPs) are described in the prior art, in which the AuNPs are functionalized with oligonucleotides and cross-linked by means of complementary oligonucleotides. These experiments exploit the known property of an AuNP solution of undergoing a visible change of colour (from red to blue) when the interparticle distance decreases.

Alivisatos P. A. et al., Organization of “Nanocrystal Molecules” Using DNA, Nature 1996, 6592, 609-611 describes the formation of dimers and trimers of colloidal gold nanoparticles (AuNPs) functionalized with a single-stranded oligonucleotide, through the use of a complementary oligonucleotide.

In Mirkin C. A. et al. A DNA-Based Method for Rationally Assembling Nanoparticles into Macroscopic Materials, Nature 1996, 6592, 607-609, a method is described which provides for the functionalization of two series of AuNPs with non-complementary DNA oligonucleotides bearing thiol groups, which bind to gold. A duplex oligonucleotide with free single-stranded ends (“sticky ends”), which are complementary to the two sequences with which the AuNPs are functionalized, is then added to the AuNP solution, which causes self assembly of the AuNPs into aggregates.

The scheme outlined above requires the functionalization of two series of AuNPs with two different oligonucleotide probes, each complementary to a portion of the target DNA, in such a way that the presence of the target induces AuNP cross-linking and the consequent change of colour of the solution. This scheme is suitable for the detection of short sequences of target DNA, but cannot be used as a universal detector for the amplification of nucleic acids, for the following reasons:

1) for each target, a pair of AuNPs functionalized with oligonucleotide probes specific for the target must be prepared; 2) the change in colour (from red to blue) is greatest with very small interparticle distances, but it gradually becomes less evident as the interparticle distances increase; however, most of the PCR amplification products are longer (typically >30 base pairs) than the cross-linking oligonucleotide probes which allow a proper plasmon coupling of the AuNPs and a noticeable colour change; 3) the PCR products are double-stranded and must be denatured before hybridization with the complementary oligonucleotide probes immobilized on the AuNPs; 4) even after denaturation, the complementary strand of the target competes with the oligonucleotide probes, which causes a marked reduction in the sensitivity of the assay.

An assay based on the implementation of an asymmetric PCR and hybridization with AuNP probes is described in the scientific literature (Deng H et al. Gold Nanoparticles with Asymmetric Polymerase Chain Reaction for Colorimetric Detection of DNA Sequence, Anal. Chem. 2012, 84, 1253-1258). However, this assay only overcomes the above-mentioned disadvantages in items 3) and 4), thanks to the use of an asymmetric PCR, but it still requires a target-specific functionalization of the AuNPs and only detects moderate colour changes (from red to light purple), probably because of steric hindrance of the relatively long PCR product, which interferes with the aggregation of the AuNPs.

In order to overcome these and other drawbacks of the prior art, the inventors developed methods and related kits as defined in the appended independent claims.

Specific embodiments of the invention are the subject of the dependent claims, the content of which is to be understood as an integral part of the description.

The invention which is the object of the present patent application solves the problems of the prior art by providing a universal detection system for nucleic acid amplification which requires neither specific equipment, nor long execution times, nor the design of oligonucleotide probes specific for the target.

Advantageously, the methods and kits of the invention provide for rapid detection of the amplification product (amplicon), which can be carried out with the naked eye generally within 3-20 minutes and requires no specific instruments or further sample manipulations. The operation is similar to that of a pH indicator. In the most simple embodiment of the invention, in fact, it is sufficient to add a small aliquot of the PCR reaction in a test tube containing a red solution and observe the result after approximately 3 minutes. If the solution remains red, this means that no amplicons, and therefore no targets, were present. On the contrary, if the PCR reaction contains the amplicon, the solution becomes violet/blue. This one-step one-tube procedure can also be performed by non-specialized personnel. If coupled with portable equipment and automated for PCR, it can be easily implemented in any context, even where there is no available laboratory equipment.

Further features and advantages of the invention will become apparent from the detailed description which follows, given purely by way of non-limiting example, with reference to the accompanying drawings, in which:

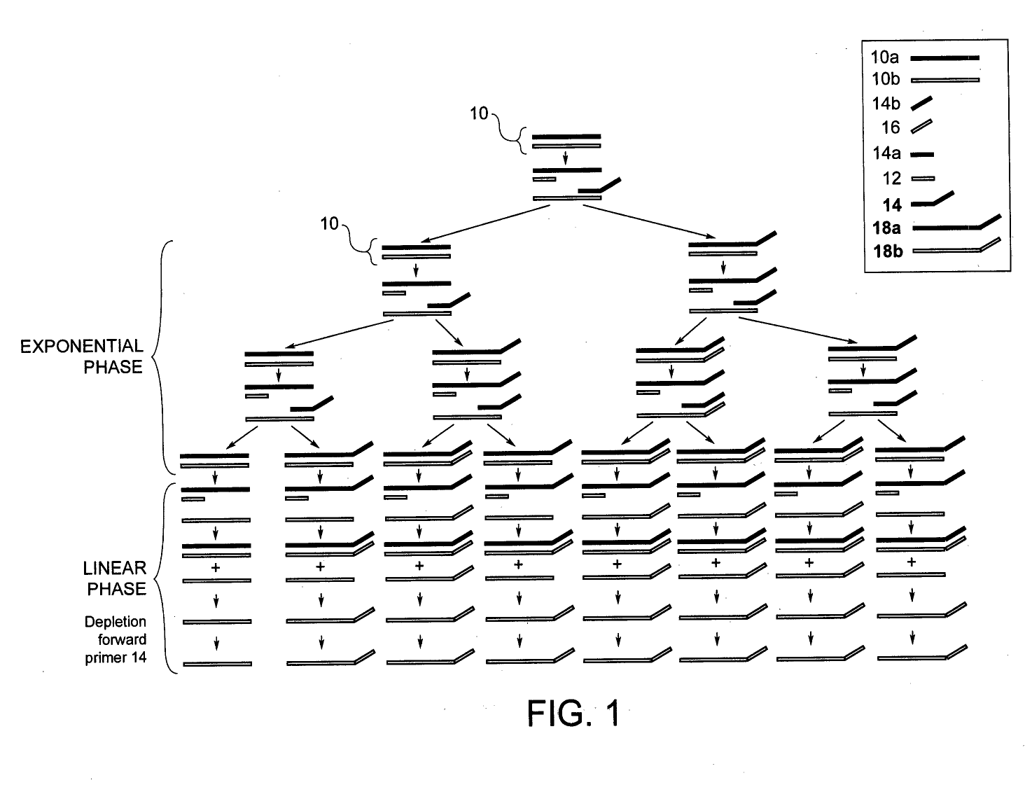

FIG. 1 is a diagram illustrating an asymmetric PCR amplification using a forward primer including at its 5′ end a universal tag sequence (TAG) which is incorporated in the PCR amplification product;

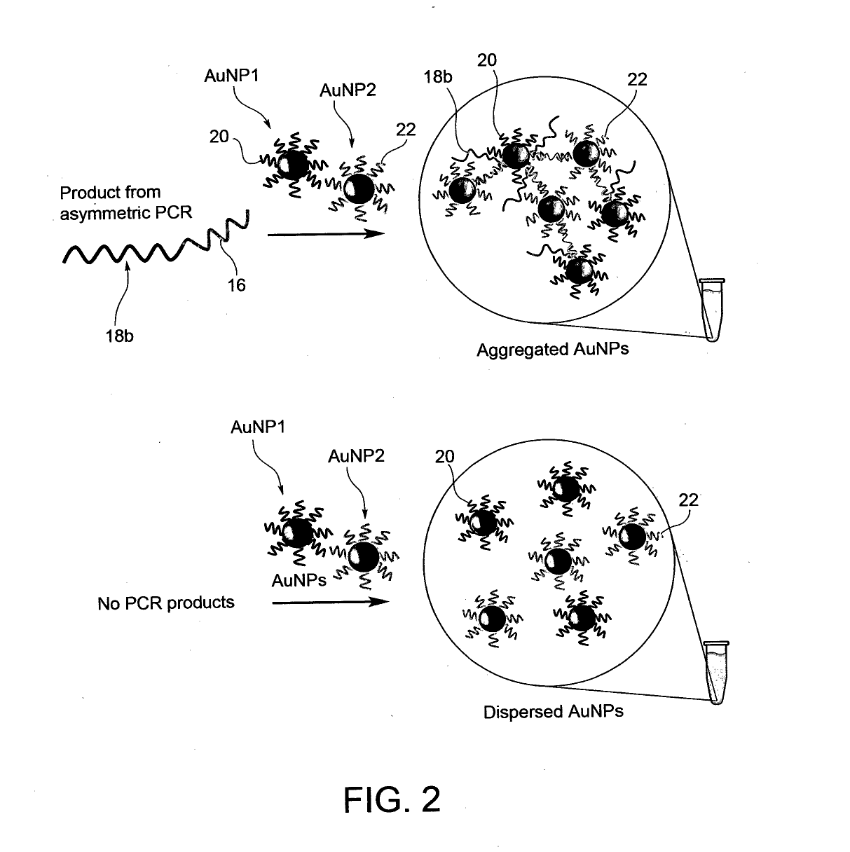

FIG. 2 is a diagram illustrating a first embodiment of the detection method of the invention;

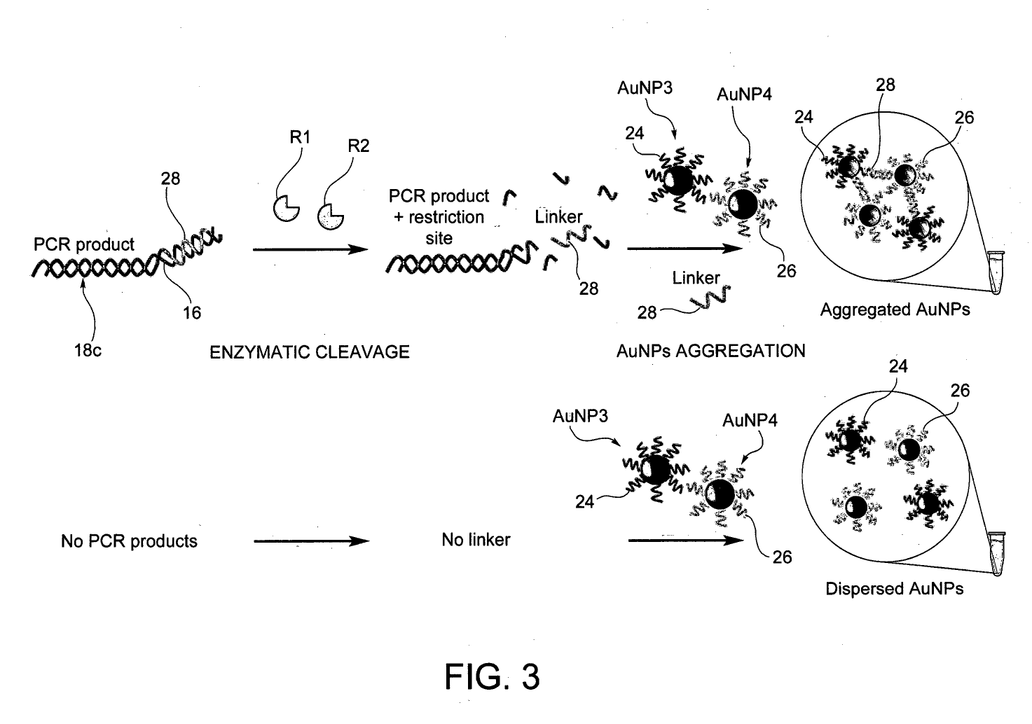

FIG. 3 is a diagram illustrating a second embodiment of the detection method of the invention;

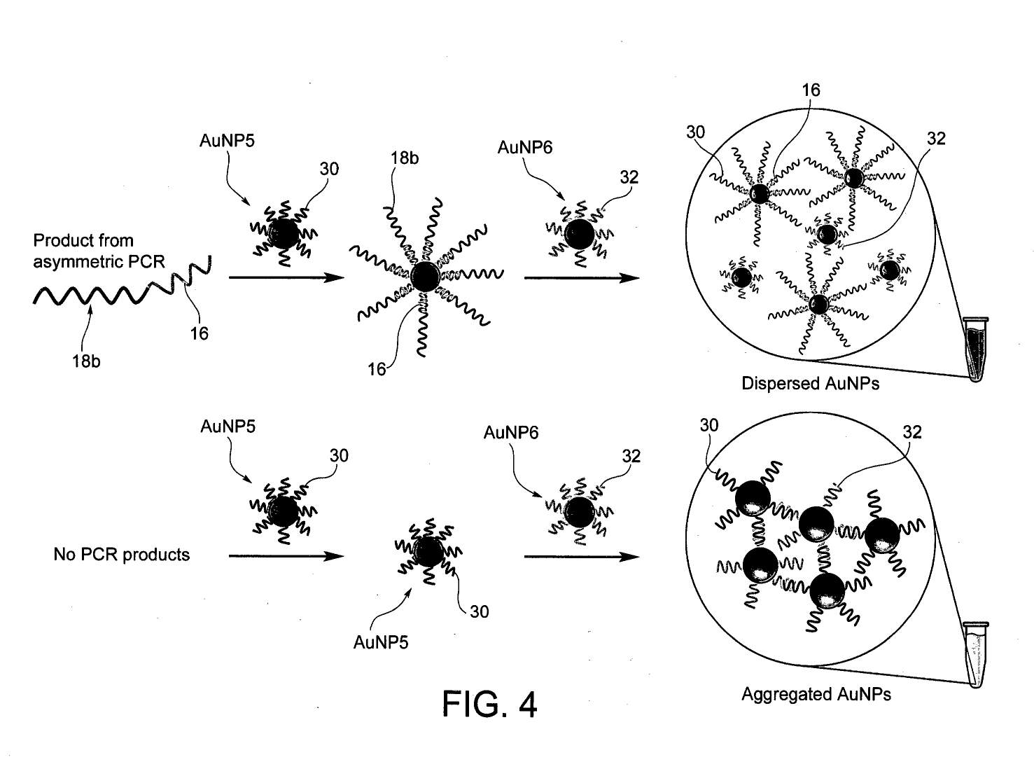

FIG. 4 is a diagram illustrating a third embodiment of the detection method of the invention;

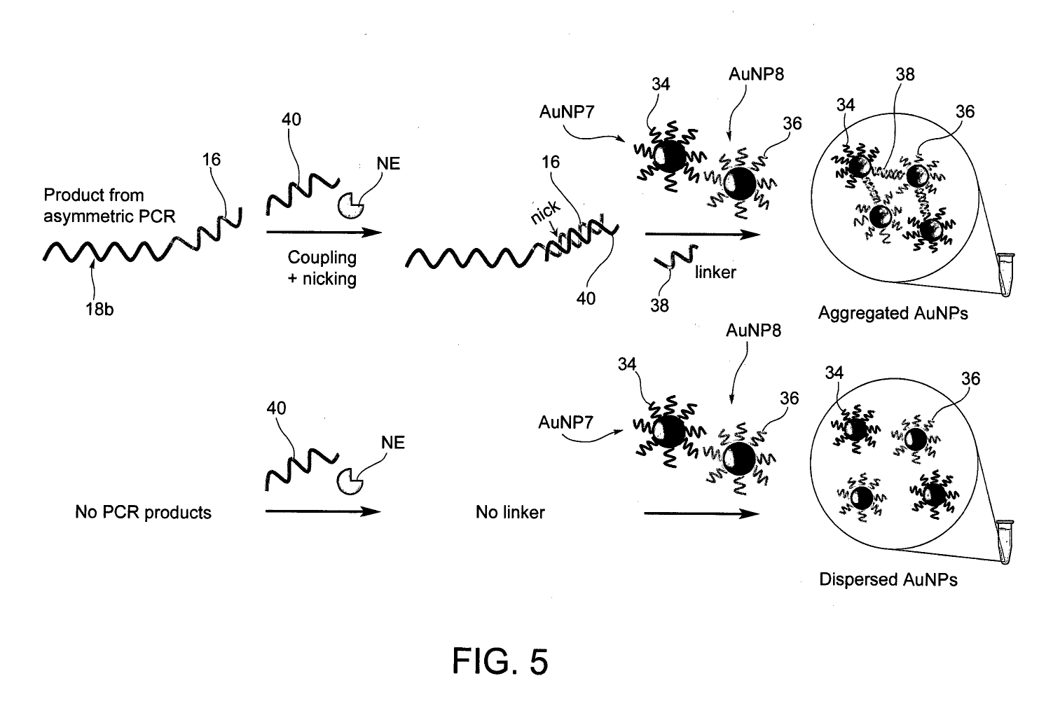

FIG. 5 is a diagram illustrating a fourth embodiment of the detection method of the invention;

The method of the invention is performed on a sample resulting from a nucleic acid amplification reaction capable of generating a single-stranded, or alternatively a double-stranded, amplification product (also designated as “amplicon”), in which a universal tag sequence is incorporated (TAG). A single stranded amplicon incorporating a TAG is preferably obtained by asymmetric PCR; a double stranded amplicon incorporating a TAG is preferably obtained by standard PCR.

An asymmetric PCR reaction resulting in the incorporation of a universal tag sequence (TAG) in the amplification product is schematically illustrated in FIGS. 1.

10a and 10b, respectively, indicate the coding strand and the non-coding strand of the target nucleic acid 10, i.e. the nucleic acid region that is to be amplified and detected. The target DNA generally has a length of between approximately 30 and approximately 100 base pairs.

The amplification is performed by means of a reverse primer 12 that hybridizes to the coding strand 10a of the target 10 and a forward primer 14 that hybridizes to the non-coding strand 10b of the target 10. The forward primer 14 comprises a region binding the target 14a, consisting of a nucleic acid sequence designed to hybridize with the non-coding strand 10b, and a region 14b that is complementary to a universal tag sequence 16, the said region 14b not being capable of hybridizing to the target. The region 14b is located at the 5′ end of the forward primer 14. As shown in FIG. 1, in the course of the amplification reaction, the region 14b is incorporated in the amplification product 18a of the target nucleic acid 10, and the universal tag sequence 16 (which is complementary to region 14b ) is incorporated in the amplification product 18b complementary to the amplification product 18a. Since in an asymmetric PCR the reverse primer 12 is used in excess over the forward primer 14 (the molar ratio of forward:reverse being generally between 1:5 and 1:1000, preferably between 1:20 and 1:50), in the last amplification cycles the forward primer 14 is depleted and the amplification is not exponential anymore. Consequently, at the end of the asymmetric PCR reaction, an accumulation of amplification product 18b occurs, which includes the universal tag sequence 16 at one of its ends. FIG. 1 shows that the universal tag sequence 16 is not present at the beginning of the amplification reaction and is not synthesized in the absence of target nucleic acid 10. It therefore represents a universal indicator of the presence of the target 10 in the sample.

After the amplification reaction, the universal tag sequence 16 is detected by means of a method that constitutes the object of the present invention.

FIG. 2 shows a first embodiment of the detection method of the invention, wherein the detection is carried out via a first colloidal gold nanoparticle AuNP-1 functionalized with a first oligonucleotide probe 20 and a second colloidal gold nanoparticle AuNP-2 functionalized with a second oligonucleotide probe 22. The first oligonucleotide probe 20 is complementary to a first portion of the universal tag sequence 16 and the second oligonucleotide probe 22 is complementary to a second portion of the universal tag sequence 16. To detect the success of the amplification colorimetrically, a premixed solution of the AuNP-1 and AuNP-2 colloidal gold nanoparticles is added to one aliquot of the sample subjected to asymmetric PCR illustrated in FIG. 1. In the presence of target nucleic acid 10, the amplification reaction leads to the synthesis of the amplification product 18b including the universal tag sequence 16. This causes the cross-linking of the AuNP-1 and AuNP-2 colloidal gold nanoparticles via the respective oligonucleotide probes 20 and 22 that hybridize to the respective portions of the universal tag sequence 16. The end result is a change in colour of the solution from red to violet/blue. On the contrary, in the absence of target nucleic acid 10, the AuNP-1 and AuNP-2 colloidal gold nanoparticles do not cross-link because the amplification product 18b including the universal tag sequence 16 does not form and, consequently, variation in the colour of the solution is not observed. This embodiment advantageously allows to detect amplification of relatively short targets in an extremely short period of time (about 2-3 minutes).

FIG. 3 shows a second embodiment of the detection method of the invention, wherein the detection is carried out via a first colloidal gold nanoparticle AuNP-3 functionalized with a first oligonucleotide probe 24 and a second colloidal gold nanoparticle AuNP-4 functionalized with a second oligonucleotide probe 26. In this embodiment, the first oligonucleotide probe 24 is complementary to a first portion of a linker sequence 28 and the second oligonucleotide probe 26 is complementary to a second portion of the linker sequence 28. The linker sequence 28 is generated by enzymatic digestion of the universal tag sequence 16, which according to this embodiment is designed in such a way as to include a first restriction site, and optionally a second restriction site (not shown in FIG. 3). The first restriction site is located at the proximal end of the universal tag sequence 16 and is able to be cleaved by a first restriction enzyme R1. The second optional restriction site is located at the distal end of the universal tag sequence 16 and is able to be cleaved by a second optional restriction enzyme R2. To detect the successful amplification of the target nucleic acid sequence colorimetrically, in a first step, the restriction enzyme or enzymes R1, R2 required to generate the linker sequence 28 and, in a second step, a premixed solution of the AuNP-3 and AuNP-4 colloidal gold nanoparticles are added to an aliquot of a sample subjected to symmetrical PCR. In the presence of target nucleic acid, the amplification reaction leads to the synthesis of a double-stranded amplification product 18c including the universal tag sequence 16, also in the double-stranded form. The latter is digested by the first and optionally the second restriction enzyme R1, R2 to give the single-stranded linker sequence 28, which in turn causes the cross-linking of the AuNP-3 and AuNP-4 nanoparticles via the respective oligonucleotide probes 24 and 26. The end result is a change in colour of the solution from red to violet/blue. On the contrary, in the absence of target nucleic acid, the AuNP-3 and AuNP-4 colloidal gold nanoparticles do not cross-link because the double-stranded amplification product 18c including the universal tag sequence 16 does not form and, consequently, the single-stranded linker sequence 28 does not form. Thus, no change in colour of the solution is observed. This embodiment advantageously allows for the detection of an amplification product of any length. It also allows for the detection of amplification products from symmetrical PCR reactions. At the end of the PCR reaction a brief digestion with the restriction enzyme or enzymes is carried out on small aliquots of the PCR product. The selected restriction enzyme or enzymes are high fidelity and fast enzymes, which allow for a complete digestion in about 10 minutes. In addition, they are chosen so as not to be affected by the components of the PCR buffer. Since this embodiment is particularly indicated for cloning procedures, the restriction enzymes are preferably selected from among the most widely used in these procedures, so that the user can directly use the digested PCR product after the detection procedure. For example, restriction enzymes that can be used in this application are: Xhol, EcoRI, SalI, XbaI, DraIII, KpnI, PstI, NheI, AgeI.

FIG. 4 shows a third embodiment of the detection method of the invention, wherein the detection is carried out via a first colloidal gold nanoparticle AuNP-5 functionalized with a first oligonucleotide probe 30 and a second colloidal gold nanoparticle AuNP-6 functionalized with a second oligonucleotide probe 32. The first oligonucleotide probe 30 is complementary to a first portion of the universal tag sequence 16 and the second oligonucleotide probe 32 is complementary to the first oligonucleotide probe 30. To detect the success of the amplification colorimetrically, a solution of the AuNP-5 colloidal gold nanoparticles and then a solution of the AuNP-6 colloidal gold nanoparticles are added to an aliquot of the sample subjected to asymmetric PCR illustrated in FIG. 1. In the presence of target nucleic acid 10, the amplification reaction leads to the synthesis of the amplification product 18b including the universal tag sequence 16, which hybridizes with the oligonucleotide probe 30 of the AuNP-5 nanoparticles, forming a structure stabilized by steric effects. The subsequent hybridization with the oligonucleotide probe 32 of the AuNP-6 particles is thus inhibited. Therefore, in the presence of the amplification product 18b, the solution remains red. On the contrary, in the absence of the amplification product 18b, the AuNP-5 and AuNP-6 nanoparticles aggregate, thanks to the hybridization of the oligonucleotide probe 30, with the complementary oligonucleotide probe 32 and therefore a change in colour can be observed. This embodiment advantageously allows for the detection of target nucleic acid sequences of any length with a very fast procedure.

FIG. 5 shows a fourth embodiment of the detection method of the invention, wherein the detection is carried out via a first colloidal gold nanoparticle AuNP-7 functionalized with a first oligonucleotide probe 34 and a second colloidal gold nanoparticle AuNP-8 functionalized with a second oligonucleotide probe 36. The first oligonucleotide probe 34 is complementary to a first portion of a linker sequence 38 and the second oligonucleotide probe 36 is complementary to a second portion of the linker sequence 38. The linker sequence 38 is generated by enzymatic digestion of the universal tag sequence 16, which according to this embodiment is designed in such a way as to include a restriction site for a nicking endonuclease NE. To detect the successful amplification of the target nucleic acid sequence colorimetrically, in a first step, the nicking endonuclease NE and a single-stranded sequence 40 complementary to a portion of the universal tag sequence 16 are added to an aliquot of a sample subjected to asymmetric PCR. In a second step, a premixed solution of the AuNP-7 and AuNP-8 colloidal gold nanoparticles is added. In the presence of target nucleic acid, the amplification reaction leads to the synthesis of the single-stranded amplification product 18b including the universal tag sequence 16. The complementary sequence 40 hybridizes to the universal tag sequence 16, forming a partially single-stranded and partially double-stranded nucleic acid hybrid, which is cut by the nicking endonuclease NE, thus generating the linker sequence 38. The linker sequence 38 causes the cross-linking of the AuNP-7 and AuNP-8 nanoparticles via the respective oligonucleotide probes 34 and 36. The end result is a change in colour of the solution from red to violet/blue. On the contrary, in the absence of target nucleic acid, the AuNP-7 and AuNP-8 colloidal gold nanoparticles do not cross-link because the amplification product 18b including the universal tag sequence 16 does not form and, consequently, the single-stranded linker sequence 38 does not form. Thus, no change in colour of the solution is observed. This embodiment allows for the detection of target nucleic acid sequences of any length.

In the above-described embodiments, suitable to be used for the detection of products from asymmetric or symmetric PCR, the PCR negative control sample also acts as a control for the detection method, as it contains all the reaction constituents with the only exception of the universal tag sequence 16.

Importantly, all the embodiments of the detection method of the invention employ a universal detector, i.e., the universal tag sequence 16, and are therefore also suitable for use in multi-well formats for simultaneous detection of a plurality of samples, even if they contain different target sequences. In the case of detection of different target sequences, as a preliminary control only the first time that the amplification is performed, it is sufficient to check on a gel that the amplification product is of the expected length and that there are no unspecific products.

The size (diameter) of the AuNP colloidal gold particles used in the detection methods according to the invention is generally comprised between 1 nm and 500 nm, preferably between 15 nm and 80 nm. Their density of functionalization with the oligonucleotide probes is preferably comprised between 2×10⁻⁴ and 2×10⁻¹/nm², more preferably between 1×10⁻³/nm² and 8×10⁻²/nm².

In some embodiments, the oligonucleotide probes may include a spacer sequence that is not complementary to the universal tag sequence 16 and that serves to optimize the efficiency of the reaction. The total length of the oligonucleotide probes may range from 5 nucleotides (nt) to 80 nt, preferably from 15 nt to 40 nt. The length of the universal tag sequence 16 may range from 5 nt to 60 nt, preferably from 8 nt to 30 nt; further preferred lengths are 5 nt to 30 nt, 8 to 60 nt, 10 to 40 nt, 5 to 35 nt.

A preferred minimum length is 5, 6, 7, 8, 9 or 10 nucleotides. A preferred maximum length is 30, 35, 40, 50, 55 or 60 nucleotides. All the above minimum and maximum length values can be combined with each other to give a preferred length range.

It should be noted that a limited length of the tag, which is shorter than the typical short amplification product of a PCR (amplicon), causes a significant and advantageous increase in the sensitivity of the assay. The use of short universal tag sequences also allows for a more rapid colour change, which is critical for rapid and accurate detection to the naked eye.

It should also be noted that, although in principle colloidal gold particles functionalized with oligonucleotides complementary to respective regions of the target could hybridize at internal positions of the amplicon (in order to reduce the inter-particle distance and therefore maximize plasmon coupling), in practice this is not feasible because of the steric hindrance of the terminal regions of the amplicon. The free ends of the amplicon interfere with the hybridization of the AuNPs in the internal regions of the amplicon.

Preferred examples of the universal tag sequence 16 are:

(SEQ ID NO: 1)
TAAACTCTGATGTA
(SEQ ID NO: 2)
AAACTCTGATGT
(SEQ ID NO: 3)
AACTCTGATG
ACTCTGATG

Preferred examples of the first oligonucleotide probe are:

(SEQ ID NO: 4)
5′ TTTTTATCATCATACATCA 3′;
(SEQ ID NO: 5)
5′ TTTTTATCATCTGTACCTG 3′;
(SEQ ID NO: 6)
5′ ACATCAGAGTCAACATTTTTTTTTT 3′.

Preferred examples of the second oligonucleotide probe are:

(SEQ ID NO: 7)
5′ GAGTTTACCAAGTATTTTTTTTTT 3′;
(SEQ ID NO: 8)
5′ GTGAATTACAAGTATTTTT 3′;
(SEQ ID NO: 9)
5′ TGTTGACTCTTTTTTTTTTT 3′.

In the experimental section that follows, which is provided by way of illustration only, detection experiments are described which were carried out using the beta-actin gene as the target nucleic acid sequence. For the embodiment shown in FIG. 2, an asymmetric PCR was performed with a primer pair that amplifies a relatively short target of 35 nucleotides. The reaction product was preliminarily analyzed by electrophoresis to confirm the presence of bands of the expected size. A small aliquot of the reaction product was then added to appropriately functionalized colloidal gold nanoparticles. For the embodiment shown in FIG. 3, a symmetric PCR was carried out on a longer fragment of the beta-actin gene, and then an enzymatic digestion was performed for 10 minutes. A small aliquot of the enzymatic digestion reaction was then added to a mixture of appropriately functionalized colloidal gold nanoparticles. For the embodiment shown in FIG. 4, an asymmetric PCR was performed and a first type of appropriately functionalized colloidal gold nanoparticles, and then a second type of gold nanoparticles functionalized with a complementary oligonucleotide, were added to a small aliquot of the reaction product. In all cases, the amplification reaction product could be detected with the naked eye in a few minutes.

### EXAMPLES

### Example 1

An asymmetric PCR was carried out using the forward primer: ACATCAGAGTTTCCAGCACAATGAAGATCA (SEQ ID NO:10) and the reverse primer: AGGAAAGACACCCACCT (SEQ ID NO:11).

These primers amplify a portion of 35 nucleotides of the gene encoding beta actin.

The reaction mixture contained 500 nM reverse primer, 25 nM forward primer, 1 μl of Taq polymerase, 0.2 mM dNTP mix, 1× Taq reaction buffer, 2.5 mM MgCl2 and 100 ng of genomic DNA extracted from HeLa cell cultures, in a total volume of 50 μl. A sample designated as the negative control was prepared in the same way, with the exception of the genomic DNA, which was absent in the negative control. After 35 rounds of PCR, an aliquot of each reaction was run on an 18% native polyacrylamide gel to verify the formation of the single-stranded amplification product. After that, an aliquot of the asymmetric PCR reaction (from 5 to 10 μl) was added to a 1:1 mixture of colloidal gold nanoparticles functionalized with a first and a second oligonucleotide probe respectively (named AuNPs-1 and AuNPs-2 respectively). First oligonucleotide probe: 5′ TTTTTATCATCATACATCA 3′ (SEQ ID NO:4); second oligonucleotide probe: 5′ GAGTTTACCAAGTATTTTTTTTTT 3′ (SEQ ID NO:7). The AuNP-1 and AuNP-2 mixture had a final concentration of 1 nM. After the addition of the PCR product, in a few minutes a change in colour could be observed, which was caused by the cross-linking of the two types of nanoparticles on the target through the universal tag sequence.

### Example 2

A standard symmetric PCR was carried out using the following primers: forward: 5′ TTGGTACCTGGTGAATTCCTTCCCTCCTCAGATCATTG 3′ (SEQ ID NO:12) and reverse: 5′ GATCCACACGGAGTACTTG 3′ (SEQ ID NO:13).

These primers amplify a portion of 52 nucleotides (nt) of the gene encoding beta actin.

The reaction mixture contained 500 nM reverse primer, 500 nM forward primer, 1 μl of Taq polymerase, 0.2 mM dNTP mix, 1× Taq reaction buffer, 2.5 mM MgCl₂ and 100 ng of genomic DNA extracted from HeLa cell cultures, in a total volume of 50 μl. A sample designated as the negative control was prepared in the same way, with the exception of the genomic DNA, which was absent in the negative control. After 35 rounds of PCR, an aliquot of each reaction was run on an 18% denaturing polyacrylamide gel to verify the formation of the amplification product of the desired length. A small aliquot of the PCR reaction (from 5 to 10 μl) was mixed with 1 μl of EcoRI-HF and 1 μl of Kpnl-HF, in the presence or absence of the specific enzyme buffer, and incubated for 10 minutes at 37° C. Thereafter, an aliquot (from 5 to 10 μl) of the enzymatic digestion reaction was added to a 1:1 mixture of colloidal gold nanoparticles functionalized with two separate oligonucleotide probes (referred to respectively as AuNP-3 and AuNP-4). AuNP-3 oligonucleotide probe: 5′ TTTTTATCATCTGTACCTG 3′ (SEQ ID NO:5); AuNP-4 oligonucleotide probe: 5′ GTGAATTACAAGTATTTTT 3′ (SEQ ID NO:8). The AuNP-3 and AuNP-4 mixture had a final concentration of 1 nM. After a few minutes from the addition of the PCR product, a change in colour could be observed, which was caused by the cross-linking of the two types of nanoparticles through the linker formed by digestion of the amplification product with the restriction enzymes.

### Example 3

An asymmetric PCR was carried out using the forward primer 5′ ACATCAGAGTCACTTCCCTCCTCAGATCATTG 3′ (SEQ ID NO:14) and the reverse primer 5′ GATCCACACGGAGTACTTG 3′ (SEQ ID NO:15).

These primers amplify a portion of 52 nt of the gene encoding beta actin.

The reaction mixture contained 500 nM reverse primer, 25 nM forward primer, 1 μl of Taq polymerase, 0.2 mM dNTP mix, 1× Taq reaction buffer, 2.5 mM MgCl₂ and 100 ng of genomic DNA extracted from HeLa cell cultures, in a total volume of 50 μl. A sample designated as the negative control was prepared in the same way, with the exception of the genomic DNA, which was absent in the negative control. After 35 rounds of PCR, one aliquot of each reaction was run on an 18% native polyacrylamide gel to verify the formation of the single-stranded amplification product. Thereafter, one aliquot (from 5 to 10 μl) of the asymmetric PCR reaction was added to AuNP-5 nanoparticles functionalized with the oligo probe 5′ ACATCAGAGTCAACATTTTTTTTTT 3′ (SEQ ID NO:6) and incubated for 10 minutes. Then, AuNP-6 nanoparticles functionalized with the oligo probe 5′ TGTTGACTCTTTTTTTTTTT 3′ (SEQ ID NO:9) were added to the mixture. The final concentration of AuNP-5 and AuNP-6 was 1 nM. After a few minutes a change in colour could be observed, which in this case indicated the absence of the target sequence (thus, it was only seen in the negative control).

### LEGENDA DELLE TAVOLE DEI DISEGNI

TAVOLA 1/5
“FASE ESPONENZIALE” =EXPONENTIAL PHASE
“FASE LINEARE” =LINEAR PHASE
“Deplezione primer forward 14” =Depletion forward primer 14
TAVOLA 2/5
“Prodotto PCR asimmetrica” =Product from asymmetric PCR
“AuNPs aggregate” =Aggregated AuNPs
“No prodotto PCR” =No PCR products
“AuNPs disperse” =Dispersed AuNPs
TAVOLA 3/5
“AuNPs aggregate” =Aggregated AuNPs
“AuNPs disperse” =Dispersed AuNPs
“AGGREGAZIONE AuNPs” =AuNP aggregation
“Prodotto PCR +sito di restrizione” =PCR product +restriction site
“TAGLIO ENZIMATICO” =ENZYMATIC CLEAVAGE
“Prodotto PCR” =PCR product
“No prodotto PCR” =No PCR products
TAVOLA 4/5
“AuNPs disperse” =Dispersed AuNPs
“AuNPs aggregate” =Aggregated AuNPs
“Prodotto PCR asimmetrica” =Product from asymmetric PCR
“No prodotto PCR” =No PCR products
TAVOLA 5/5
“AuNPs aggregate” =Aggregated AuNPs
“AuNPs disperse” =Dispersed AuNPs
“Appaiamento +nicking” =Coupling +nicking
“Prodotto PCR asimmetrica” =Product from asymmetric PCR
“No prodotto PCR” =No PCR products

1. A method for detecting the amplification of a target nucleic acid sequence (10) in a sample resulting from a nucleic acid amplification reaction capable of generating a single-stranded amplification product (18b) comprising at one end a universal tag sequence (16) having a length of between 5 and 60 nucleotides, the method being characterized in that it comprises the steps of:
adding to said sample colloidal gold nanoparticles (AuNP1, AuNP3, AuNP5, AuNP7) functionalized with a first oligonucleotide probe (20, 24, 30, 34) and colloidal gold nanoparticles (AuNP2, AuNP4, AuNP6, AuNP8) functionalized with a second oligonucleotide probe (22, 26, 32, 36), wherein said first oligonucleotide probe is at least partially complementary to a first portion of said universal tag sequence (16) and said second oligonucleotide probe is at least partially complementary to a second portion of said universal tag sequence (16) or said second oligonucleotide probe is at least partially complementary to said first oligonucleotide probe;
detecting the possible colour change of the sample as a result of the addition of said functionalized colloidal gold nanoparticles (AuNP1, AuNP3, AuNP5, AuNP7; AuNP2, AuNP4, AuNP6, AuNP8),
wherein, when the second oligonucleotide probe is at least partially complementary to the second portion of the universal tag sequence, the colour change of the sample is indicative of the successful amplification of the target nucleic acid sequence, whereas when the second oligonucleotide probe is at least partially complementary to the first oligonucleotide probe, the absence of colour change of the sample is indicative of the successful amplification of the target nucleic acid sequence.

2. A method for detecting the amplification of a target nucleic acid sequence (10) in a sample resulting from a nucleic acid amplification reaction capable of generating a single-stranded amplification product (18b) comprising at one end a universal tag sequence (16), said universal tag sequence (16) including a nicking endonuclease (NE) recognition site, the method being characterized in that it comprises the steps of:
adding to said sample a single-stranded oligonucleotide (40) complementary to the universal tag sequence (16);
incubating under suitable conditions so that hybridization occurs with the single-stranded amplification product (18b) possibly present in the sample, whereby a hybrid partially single-stranded and partially double-stranded nucleic acid is generated;
after said incubation, incubating the sample with said nicking endonuclease (NE), whereby a single-stranded linker sequence (38) consisting of at least one portion of the universal tag sequence (16) is released from said hybrid partially single-stranded and partially double-stranded nucleic acid possibly generated;
adding to the sample colloidal gold nanoparticles (AuNP7) functionalized with a first oligonucleotide probe (34) and colloidal gold nanoparticles (AuNP8) functionalized with a second oligonucleotide probe (36), wherein said first and second oligonucleotide probes (34, 36) are complementary to respective portions of said linker sequence (38);
detecting the possible colour change of the sample as a result of the addition of said functionalized colloidal gold nanoparticles (AuNP7, AuNP8),
wherein a colour change of the sample is indicative of the successful amplification of the target nucleic acid sequence.

3. A method for detecting the amplification of a target nucleic acid sequence (10) in a sample resulting from a nucleic acid amplification reaction capable of generating a double-stranded amplification product (18c) comprising at one end a universal tag sequence (16) including a first restriction site, and optionally a second restriction site, wherein said first restriction site is located at the proximal end of the universal tag sequence (16) and is able to be cleaved by a first restriction enzyme (R1), and wherein said second optional restriction site is located at the distal end of the universal tag sequence and is able to be cleaved by a second restriction enzyme (R2), the method being characterized in that it comprises the steps of:
adding to said sample said first restriction enzyme (R1) and optionally said second restriction enzyme (R2);
incubating under conditions suitable for an enzymatic digestion reaction brought about by said first restriction enzyme (R1) and optionally by said second restriction enzyme (R2) to occur, whereby a single-stranded linker sequence (28) consisting of at least one portion of the universal tag sequence (16) is released from the amplification product (18c) possibly present in the sample;
adding to said sample colloidal gold nanoparticles (AuNP3) functionalized with a first oligonucleotide probe (24) and colloidal gold nanoparticles (AuNP4) functionalized with a second oligonucleotide probe (26), wherein said first and second oligonucleotide probes (24, 26) are at least partially complementary to respective portions of said linker sequence (28);
detecting the possible colour change of the sample as a result of the addition of said functionalized colloidal gold nanoparticles (AuNP3, AuNP4),
wherein a colour change of the sample is indicative of the successful amplification of the target nucleic acid sequence.

4. The method according to claim 1, wherein the nucleic acid amplification reaction is an asymmetric PCR employing a forward primer (14) and a reverse primer (12), wherein the reverse primer (12) is in excess over the forward primer (14), the molar ratio between forward primer (14) and reverse primer (12) being preferably between 1:5 and 1:1000, more preferably between 1:20 and 1:50.

5. The method according to claim 3, wherein the nucleic acid amplification reaction is a symmetric PCR employing a forward primer (14) and a reverse primer (12).

6. The method according to claim 1, wherein the length of each of said first and second oligonucleotide probes (20, 24, 30, 34; 22, 26, 32, 36) with which the colloidal gold particles are functionalized is between 5 and 80 nucleotides, preferably between 12 and 40 nucleotides.

7. The method according to claim 1, wherein the length of said universal tag sequence (16) included in the nucleic acid amplification reaction product is comprised between 8 and 30 nucleotides.

8. The method according to claim 1, wherein the universal tag sequence (16) included in the nucleic acid amplification reaction product is selected from the group consisting of TAA ACT CTG ATG TA (SEQ ID NO:1), AA ACT CTG ATG T (SEQ ID NO:2), A ACT CTG ATG (SEQ ID NO:3) and ACT CTG ATG.

9. The method according to claim 1, wherein said colloidal gold nanoparticles have a particle size of between 1 and 500 nm, preferably between 15 and 80 nm.

10. The method according to claim 1, wherein said colloidal gold nanoparticles are functionalized at a functionalization density of from 2×10⁻⁴/nm² to 2×10⁻¹/nm², preferably of from 1×10⁻²/nm² to 8×10⁻²/nm².

11. A kit for detecting the amplification of a target nucleic acid sequence (10) in a sample resulting from a nucleic acid amplification reaction capable of generating an amplification product (18b, 18c) including at one end a universal tag sequence (16), the kit being characterized in that it comprises:
colloidal gold nanoparticles (AuNP1, AuNP3, AuNP5, AuNP7) functionalized with a first oligonucleotide probe (20, 24, 30, 34); and
colloidal gold nanoparticles (AuNP2, AuNP4, AuNP6, AuNP8) functionalized with a second oligonucleotide probe (22, 26, 32, 36),
wherein said first oligonucleotide probe (20, 24, 30, 34) is at least partially complementary to a first portion of said universal tag sequence (16) and said second oligonucleotide probe (22, 26, 32, 36) is at least partially complementary to a second portion of said universal tag sequence (16) or said second oligonucleotide probe (22, 26, 32, 36) is at least partially complementary to said first oligonucleotide probe (20, 24, 30, 34), characterised in that it also comprises a nicking endonuclease (NE) and a single-stranded oligonucleotide (40) complementary to the universal tag sequence (16), wherein the universal tag sequence (16) includes a recognition site for said nicking endonuclease (NE).

12. A kit for detecting the amplification of a target nucleic acid sequence (10) in a sample resulting from a nucleic acid amplification reaction capable of generating an amplification product (18b, 18c) including at one end a universal tag sequence (16), the kit being characterized in that it comprises:
colloidal gold nanoparticles (AuNP1, AuNP3, AuNP5, AuNP7) functionalized with a first oligonucleotide probe (20, 24, 30, 34);
colloidal gold nanoparticles (AuNP2, AuNP4, AuNP6, AuNP8) functionalized with a second oligonucleotide probe (22, 26, 32, 36);
wherein said first oligonucleotide probe (20, 24, 30, 34) is at least partially complementary to a first portion of said universal tag sequence (16) and said second oligonucleotide probe (22, 26, 32, 36) is at least partially complementary to a second portion of said universal tag sequence (16) or said second oligonucleotide probe (22, 26, 32, 36) is at least partially complementary to said first oligonucleotide probe (20, 24, 30, 34), characterised in that it also comprises a first restriction enzyme (R1) designed to cleave the universal tag sequence (16) at a first restriction site, and optionally a second restriction enzyme (R2) designed to cleave the universal tag sequence (16) at a second restriction site, the universal tag sequence (16) including said first restriction site and optionally said second restriction site.

13. The kit according to claim 11, wherein the length of each of said first and second oligonucleotide probes (20, 24, 30, 34; 22, 26, 32, 36) with which the colloidal gold particles are functionalized is between 5 and 80 nucleotides, preferably between 15 and 40 nucleotides.

14. The kit according to claim 11, wherein said colloidal gold nanoparticles have a particle size of between 1 and 500 nm, preferably between 15 and 80 nm.

15. The kit according to claim 11, wherein said colloidal gold nanoparticles functionalized with a first oligonucleotide probe and said colloidal gold nanoparticles functionalized with a second oligonucleotide probe have a functionalization density of from 2×10⁻⁴ to 2×10⁻¹/nm², preferably from 1×10⁻³/nm² to 8×10⁻²/nm².


SEQUENCE LISTING (15 sequences)

SEQ ID NO: 1 - DNA, 14 bp, artificial
  universal tag sequence
taaactctgatgta

SEQ ID NO: 2 - DNA, 12 bp, artificial
  universal tag sequence
aaactctgatgt

SEQ ID NO: 3 - DNA, 10 bp, artificial
  universal tag sequence
aactctgatg

SEQ ID NO: 4 - DNA, 19 bp, artificial
  oligonucleotide probe
tttttatcatcatacatca

SEQ ID NO: 5 - DNA, 19 bp, artificial
  oligonucleotide probe
tttttatcatctgtacctg

SEQ ID NO: 6 - DNA, 25 bp, artificial
  oligonucleotide probe
acatcagagtcaacatttttttttt

SEQ ID NO: 7 - DNA, 24 bp, artificial
  oligonucleotide probe
gagtttaccaagtatttttttttt

SEQ ID NO: 8 - DNA, 19 bp, artificial
  oligonucleotide probe
gtgaattacaagtattttt

SEQ ID NO: 9 - DNA, 20 bp, artificial
  oligonucleotide probe
tgttgactcttttttttttt

SEQ ID NO: 10 - DNA, 30 bp, artificial
  forward primer
acatcagagtttccagcacaatgaagatca

SEQ ID NO: 11 - DNA, 19 bp, artificial
  reverse primer
gatccacacggagtacttg

SEQ ID NO: 12 - DNA, 32 bp, artificial
  forward primer
acatcagagtcacttccctcctcagatcattg

SEQ ID NO: 13 - DNA, 19 bp, artificial
  reverse primer
gatccacacggagtacttg

SEQ ID NO: 14 - DNA, 38 bp, artificial
  forward primer
ttggtacctggtgaattccttccctcctcagatcattg

SEQ ID NO: 15 - DNA, 17 bp, artificial
  reverse primer
aggaaagacacccacct